(19) United States
(12) Patent Application Publication
(10) Pub. No.: US 2023/0270467 A1
(43) Pub. Date: Aug. 31, 2023

(54) SPINAL BONE FASTENER ASSEMBLY FOR RECEIVING TWO SPINAL RODS

(71) Applicant: INNO4SPINE AG, Stans (CH)

(72) Inventors: Heiko KOLLER, Kufstein (AT); Dezsö János JESZENSZKY, Küsnacht (CH); Daniel HASCHTMANN, Küsnacht (CH); Tamás Fülöp FEKETE, Zürich (CH); Adriano VIGANÒ, Pfäffikon (CH)

(73) Assignee: INNO4SPINE AG, Stans (CH)

(21) Appl. No.: 18/016,306

(22) PCT Filed: Jul. 6, 2021
(86) PCT No.: PCT/IB2021/056045

§ 371 Date: Jan. 13, 2023

(30) Foreign Application Priority Data
Jul. 17, 2020 (CH) 00889/20

(51) Int. Cl. A61B 17/70; A61B 17/84

(52) U.S. Cl. CPC A61B 17/70; A61B 17/84

(57) ABSTRACT

There is proposed a bone fastener assembly comprising a bone fastener comprising a bone fastener head and a bone fastener shaft, a correction instrument and/or a locking insert, and a rod receiving head. The rod receiving head includes at least two rod receiving passages, and which can thus be connected to two rods. Moreover, the movement of the rod receiving head in relation to the bone fastener can be blocked prior to placing a rod into the rod receiving head. The proposed bone fastener assembly may be used for example for correcting larger spinal deformities.

### TECHNICAL FIELD OF THE INVENTION

The present invention relates to a spinal bone fastener assembly, which may be part of a spinal posterior rod system. The posterior rod system can form a new system for correcting severe deformities or dislocations. Some spinal surgical procedures require high correction forces, thereby often requiring very rigid spinal bone fastener assemblies, and a possibility of attaching manipulation force levering instruments. To maintain the correction during the bone fusion period, such a system preferably provides the possibility of receiving two parallel spinal rods in the spinal bone fastener assembly, thus comprising two rod receiving passages in the rod receiving head. The present invention also relates to a kit comprising the spinal bone fastener assembly, and to method of correcting a first spinal column portion in relation to a second spinal column portion, and of maintaining the correction using linked correction instruments, which are engaged into or over the spinal bone fastener.

### BACKGROUND OF THE INVENTION

In orthopaedic surgery around the spine, posterior spinal stabilisation systems are often placed to a target site to realign, correct and/or stabilise the spinal column to compensate for malalignment caused by for example degeneration of the spine, born malalignments, such as excessive lordosis, kyphosis and scoliosis, and for example trauma, such as fractures. Often the placement of a construct to correct the spinal column requires the application of high forces. In such cases, often a standard pedicle screw combined with one single rod cannot provide the needed stability. There is a risk that the initial position of the system may be lost due to slippage between the pedicle screw head and the rod receiving head. Even worse, the single rod may not withstand the loads it is carrying over time, and a material fatigue related rod fracture may occur.

A state-of-the-art pedicle screw assembly comprises a system of engaging elements that allows a surgeon to lock the rod, the rod receiving head and the pedicle screw simultaneously, by tightening a set screw or a rod fastener. Prior to this locking step, the rod receiving head is movably connected to the pedicle screw head so that is it configured to swivel and rotate. Due to this movable connection between the rod receiving head and the pedicle screw head, only limited correction forces can be applied directly to the pedicle screw. It is possible to apply forces that are directed substantially parallel to the screw axis. However, it is practically impossible to apply correction forces to the screw, such as rotational or tilting moments. Normally, the correction is made using instruments that grasp the rod receiving head, and the rod and pull both towards each other. After this step, the construct is fixated by tightening the rod fastener in place. Only then, the whole construct becomes fully stable. In many types of surgeries, for normal corrections, the previously described correction technique provides a surgeon with sufficient control to correct the spinal column.

However, in the cases of major corrections or largely displaced fractures, often the previously described technique does not provide a sufficient correction control. An intermediate locking step, rigidly locking the bone fastener head into the rod receiving head is needed. When locked, this will allow the surgeon to directly manipulate the vertebral body, by manipulating the rod receiving head. Thus, the bone fastener and the rod receiving head become one unit.

Furthermore, the ability of placing at least two rods into the rod receiving head is needed. This ability provides the required stability, and it prevents the rods from breaking. Additionally, the ability of attaching correction instruments, which would allow the surgeon to apply large correction forces, is needed. Preferably the correction instruments can be rigidly linked by a cross-bar, and they can independently (without holding by the surgeon) maintain the correction.

### SUMMARY OF THE INVENTION

It is an object of the present invention to overcome at least some of the problems associated with correcting large spinal deformities or displaced fractures using a state-of-the-art pedicle screw and state-of-the-art rod constructs. A solution is needed that allows the spinal column to be corrected and re-stabilised in a safe and controlled manner, when greater correction forces need to be applied. Furthermore, the risks of implant failure due to material fatigue or connection slippage need to be overcome.

Therefore, there is a need for an assembly that provides the ability and flexibility to lock the connection between a bone fastener (more specifically a bone fastener head) and a rod receiving head, prior to the introduction and fixation of a posterior rod. Furthermore, an assembly is needed that provides room for placement of at least two posterior rods. Moreover, an assembly is needed that can be connected to a correction instrument, thereby allowing a spinal vertebral body to be manipulated by manipulating the assembly. Moreover, a solution is needed that allows more correction instruments to be connected to maintain a correction in a hands-free manner, whilst placing a posterior rod for temporarily or final fixation.

According to a first aspect of the invention, there is provided a spinal bone fastener assembly as recited in claim 1.

The proposed bone fastener assembly comprises a rod receiving head including at least two rod receiving passages, and which can thus be connected to two rods. Moreover, the movement of the rod receiving head in relation to a bone fastener can be blocked prior to placing a posterior rod into the rod receiving head.

According to a second aspect of the invention, there is provided a kit comprising the spinal bone fastener assembly and at least one correction instrument sized and shaped to engage into one of the rod receiving passages of the bone fastener assembly.

According to a third aspect of the invention, there is provided a method of correcting a spinal column as recited in claim 25.

Other aspects of the invention are recited in the dependent claims attached hereto.

### BRIEF DESCRIPTION OF THE DRAWINGS

Other features and advantages of the invention will become apparent from the following description of non-limiting example embodiments, with reference to the appended drawings, in which:

FIGS. 1A to 1C depict an example bone fastener assembly according to a first embodiment of the present invention;

FIG. 2 depicts a bone fastener that may be used in the bone fastener assembly of FIGS. 1A to 1C;

FIGS. 3A to 3D depict the rod receiving head of the bone fastener assembly of

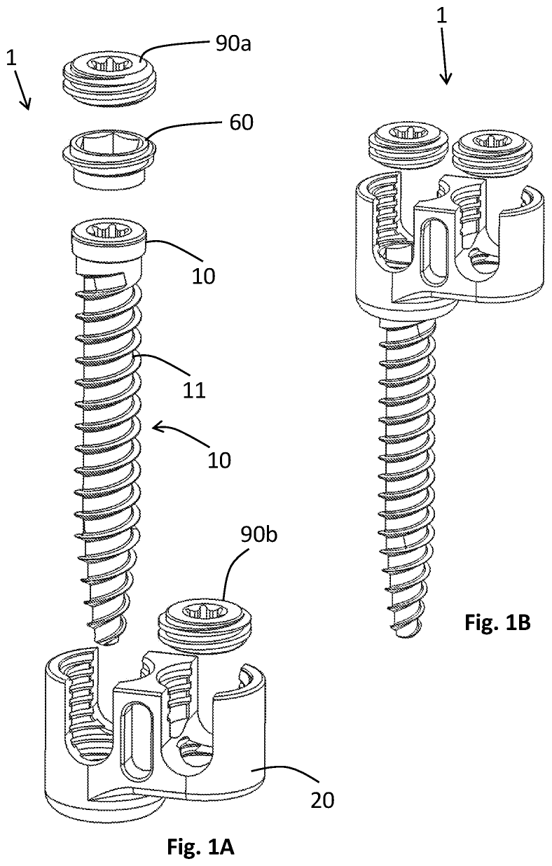

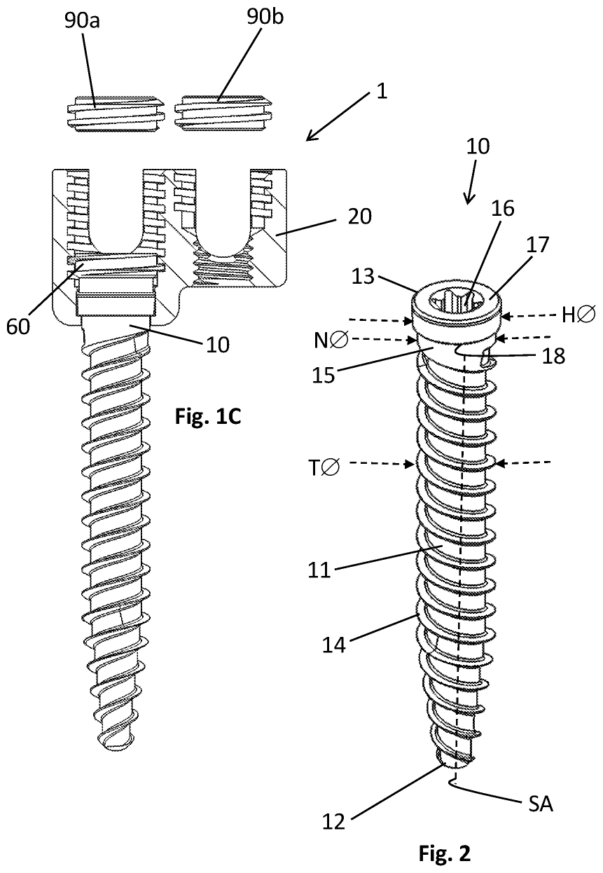

FIGS. 1A to 1C in more detail;

FIGS. 4A and 4B depict the locking insert of the bone fastener assembly of

FIGS. 1A to 1C in more detail;

FIGS. 5A and 5B depict the rod fastener of the bone fastener assembly of

FIGS. 1A to 1C in more detail;

FIGS. 6A to 6D depict a correction instrument in detail;

FIG. 7 depicts an insertion tool in detail;

FIG. 8 depicts a locking tool in detail;

FIGS. 9A to 9O depict an exemplary surgical technique using the bone fastener assembly, the correction instrument, the insertion tool and the locking tool;

FIGS. 10A to 10C depict a first locking principle;

FIGS. 11A to 11D depict an alternative design of the bone fastener assembly enabling an alternative locking principle;

FIGS. 12A to 12C depict variants of the bone fastener assembly having an extended head;

FIGS. 13A to 13C depict a variant of the correction instrument;

FIGS. 14A to 14E depict a variant of the bone fastener assembly having a rotatable rod receiving head portion;

FIGS. 15A to 15D depict a locking principle for the rotatable rod receiving head portion;

FIGS. 16A and 16B depict a variant of the rotatable rod receiving head portion including a locking arrangement; and

FIGS. 17A to 17C depict a correction instrument that can block the rotation of the rotatable rod receiving head portion.

### DETAILED DESCRIPTION OF THE INVENTION

The embodiments of the present invention will now be described in detail with reference to the attached figures. The embodiments are described in the context of placing a posterior spinal construct in its implantation location and correcting a larger deformity. Although the invention is specifically described in the context of correcting a larger deformity, the teachings of the invention are not limited to this environment. The teachings of the present invention are equally applicable to extending rod-based stabilisation constructs for other bones. When the words first and second are used to refer to different elements, it is to be understood that this does not necessarily imply or mean that the first and second elements are somehow structurally substantially different elements or that their dimensions are substantially different unless specifically stated. A bone fastener in this context means a structural element, which can be brought into the target bone, and forms a stabile connection between the target bone and the remaining spinal construct. Most often, a bone fastener is a fastening element, such as a pedicle screw. Identical or corresponding functional and structural elements which appear in the different drawings are assigned the same reference numerals.

Referring to FIGS. 1A and 1B, a spinal bone fastener assembly 1 according to an example embodiment of the present invention is shown in perspective and exploded views. FIG. 10 shows the same bone fastener assembly in a partial cross-sectional view. According to the present embodiment, the bone fastener assembly comprises a bone fastener 10, or more specifically a pedicle screw, a rod receiving head 20, which in this example is an elongated rod receiving head, a bone fastener engaging locking insert 60, and at least one rod fastener 90a, 90b, such as a set screw. The bone fastener assembly is configured to receive at least one, more preferably two state-of-the-art spinal posterior stabilisation rods, or a first rod 5 and a second rod 6, as described in greater detail later. However, the rod receiving head 20 may be configured to receive more than two rods, for example three rods. As shown in the example embodiment of FIGS. 1A to 1C, in this case the axis or direction of elongation is orthogonal or substantially orthogonal to the length or longitudinal axis of the bone fastener 10.

The bone fastener 10, which in this example is a pedicle screw, is shown in a perspective view in FIG. 2. The bone fastener comprises an elongated shaft 11 extending between the bone fastener tip 12 and a bone fastener head 13. As depicted, the bone fastener head 13 is in this example a cylindrical head, and the shaft comprises an external bone thread 14. At a transition region, where the elongated shaft 11 transitions into the bone fastener head 13, a neck section 15 is present. The bone fastener head further comprises a first drive 16 for engagement with a first insertion tool 120, such as a screwdriver. The bone fastener head with head diameter H∅ comprises a top face 17, and a bottom face 18, defining the bone fastener head thickness HT. The elongated shaft 13 defines a neck diameter N∅, a bone thread diameter T∅, and a shaft axis SA extending longitudinally across the shaft or the entire bone fastener. In the 20 present example, the bone fastener 10 is mono-axial configured to rotate only around the shaft axis SA.

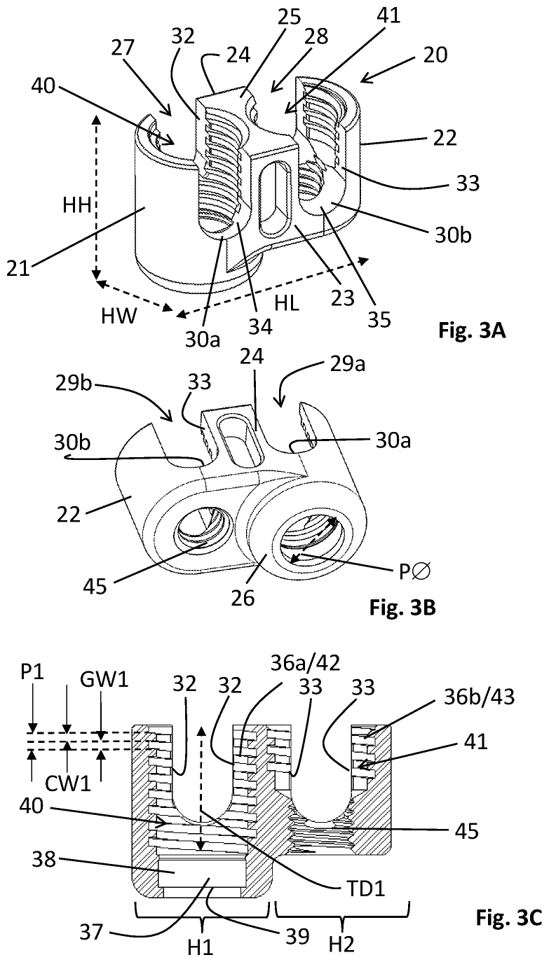

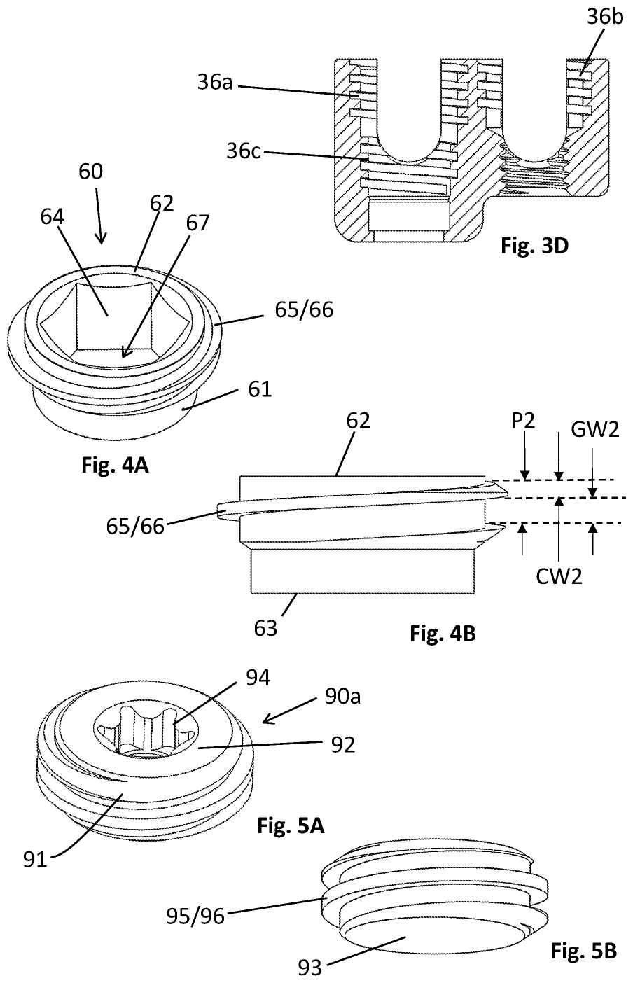

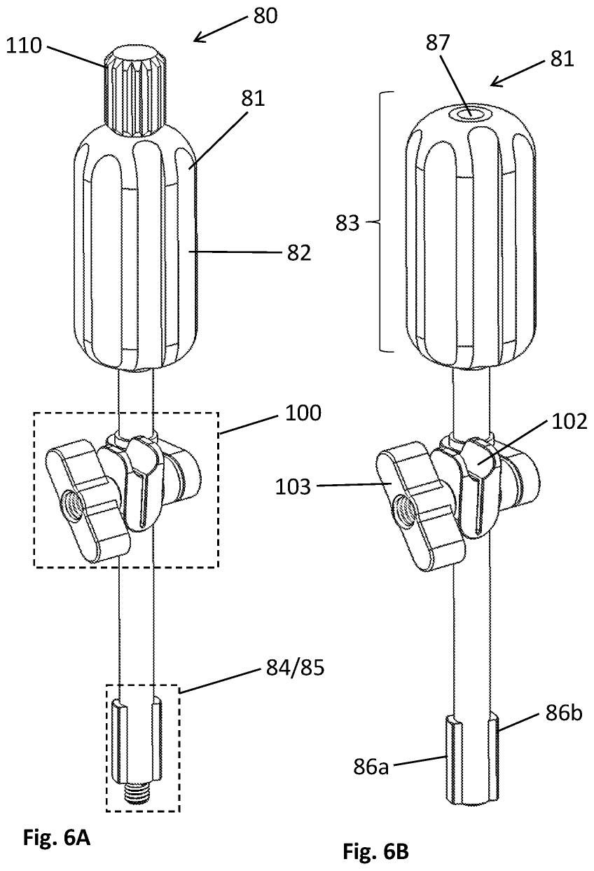

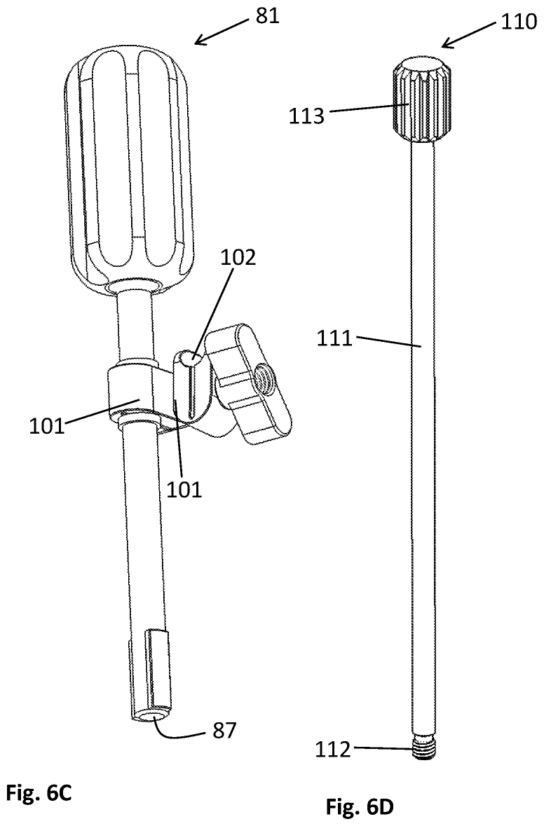

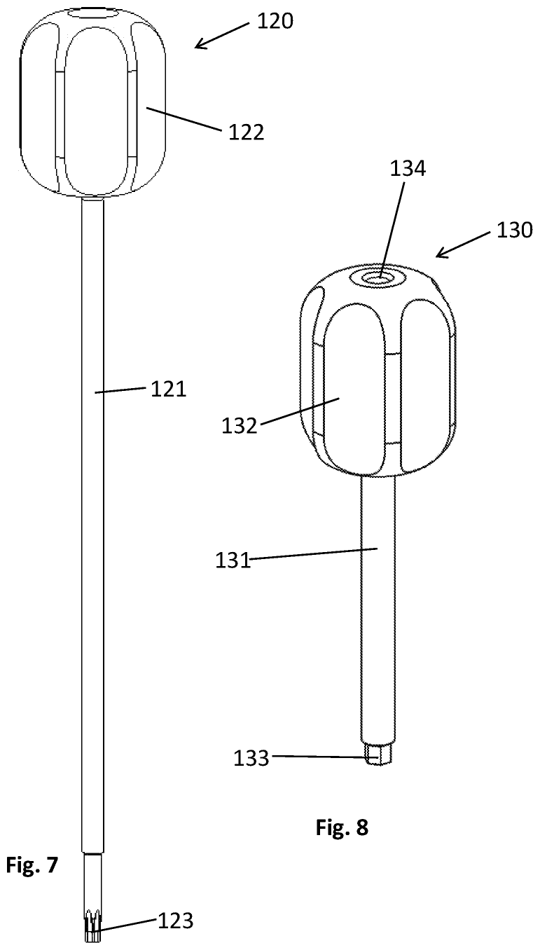

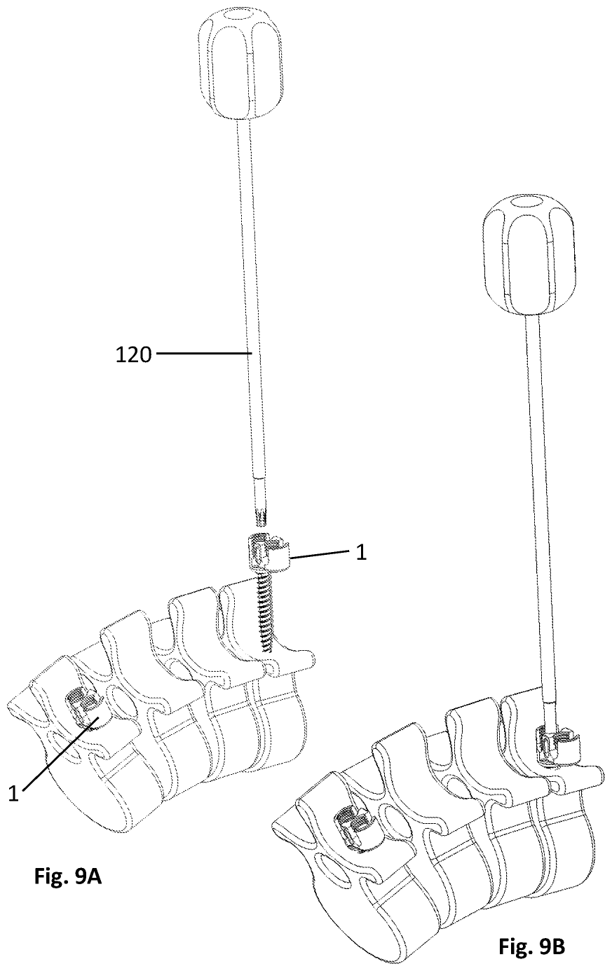

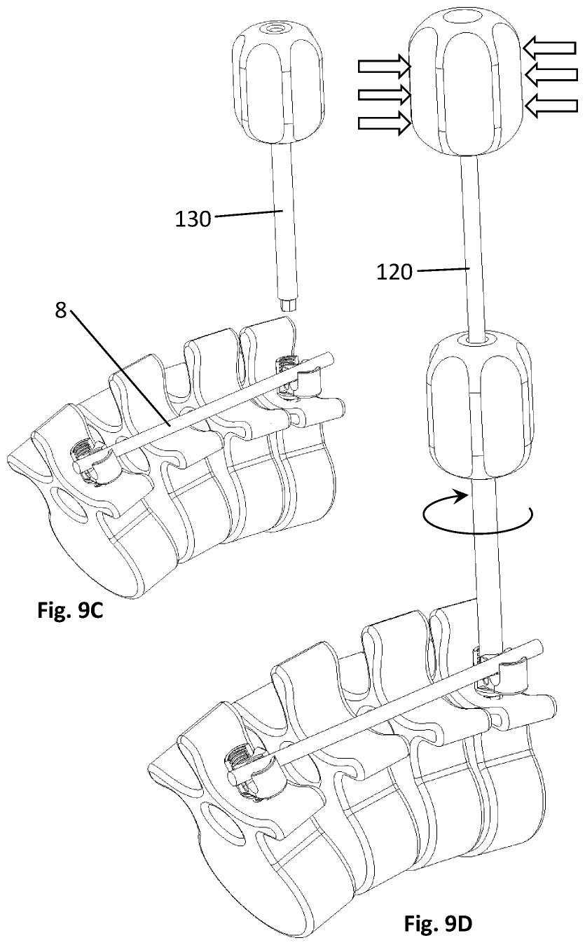

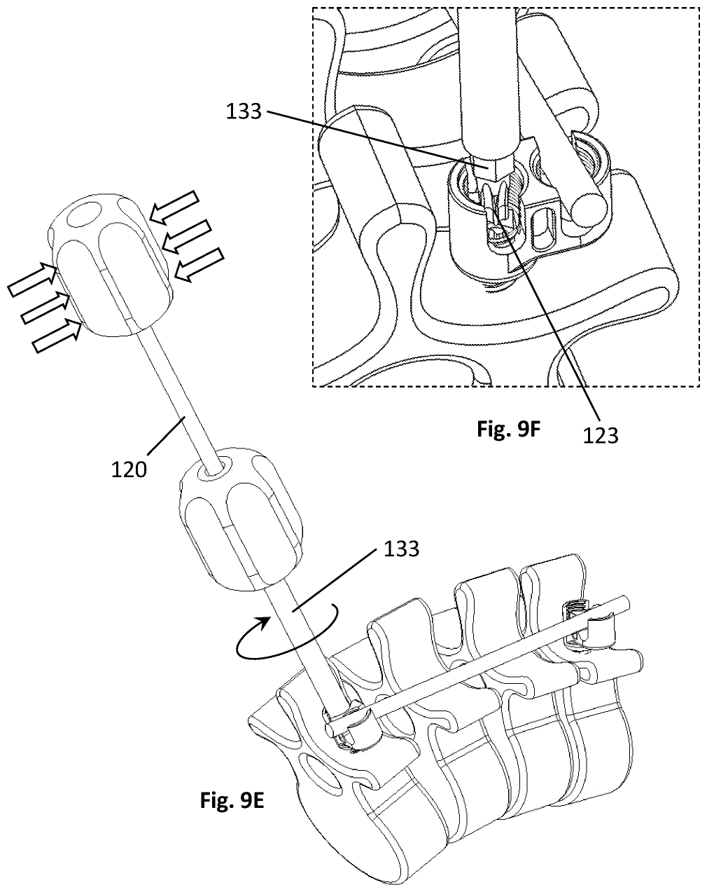

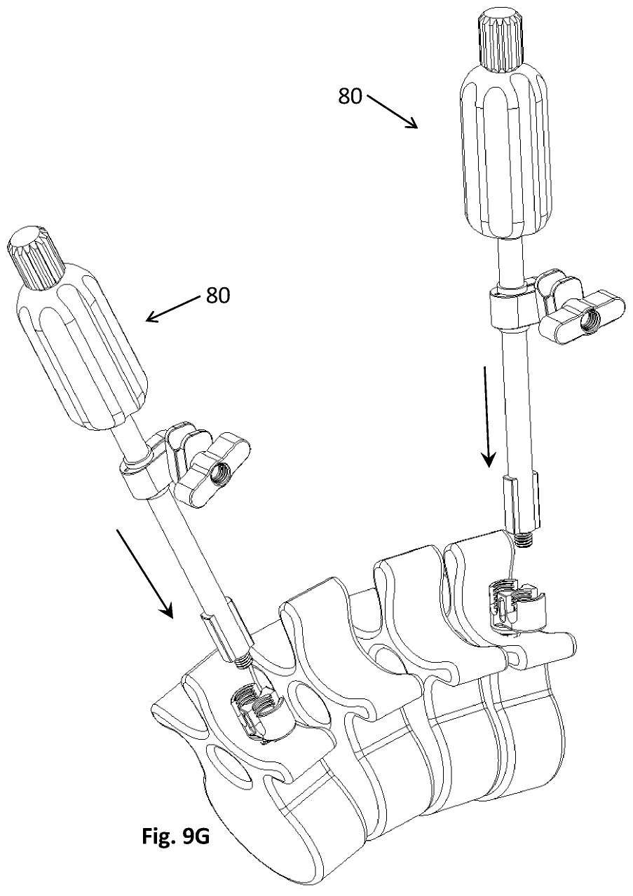

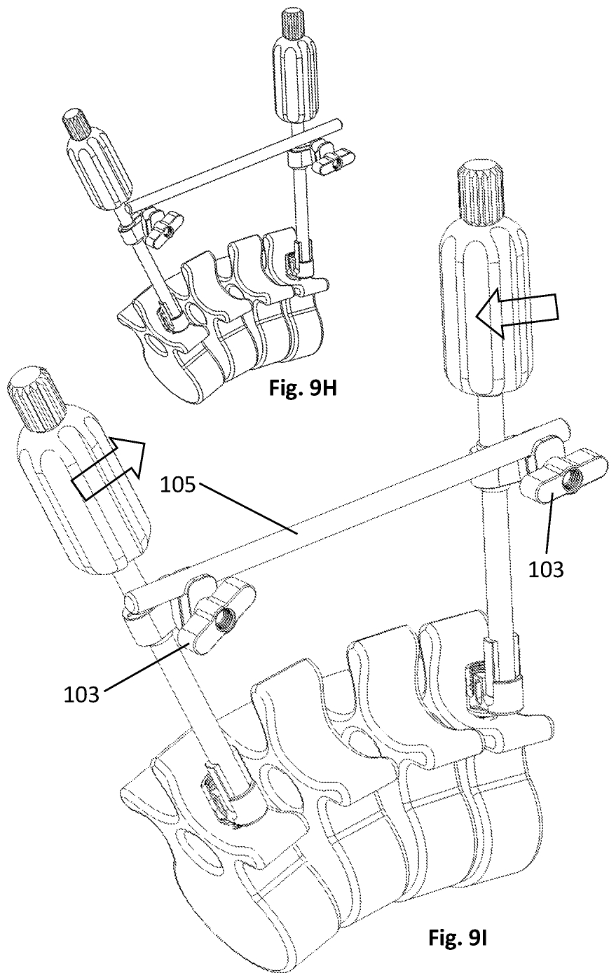

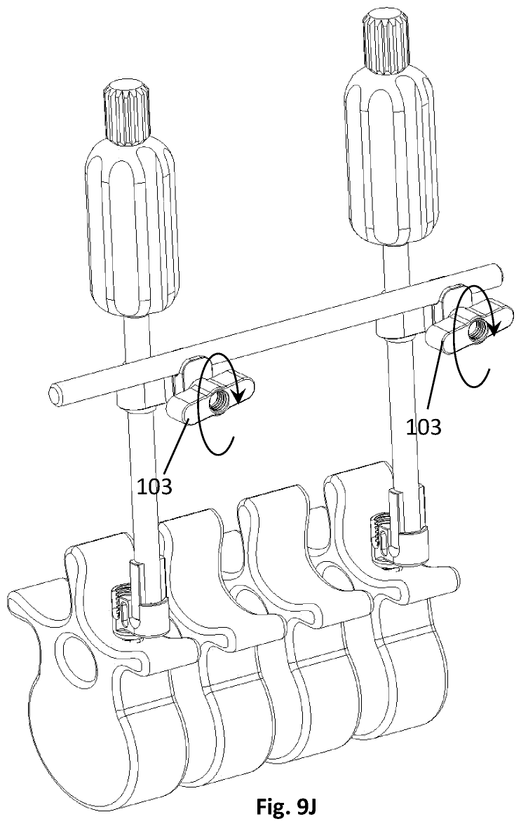

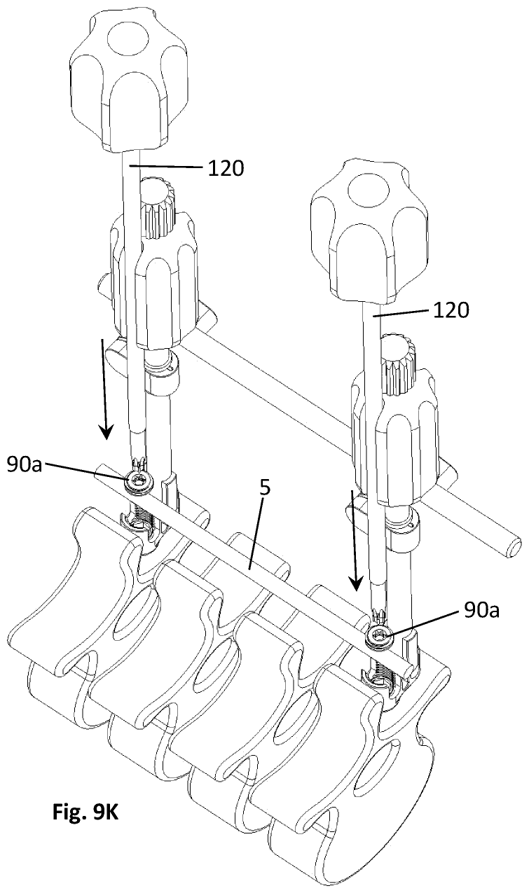

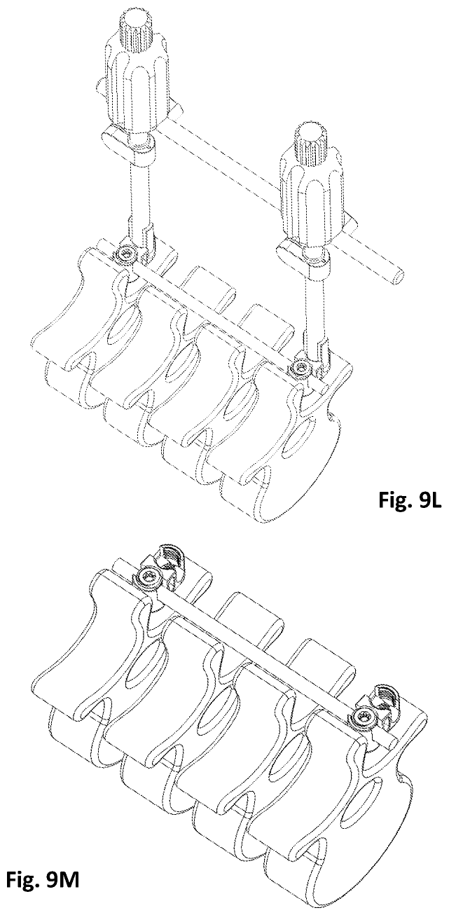

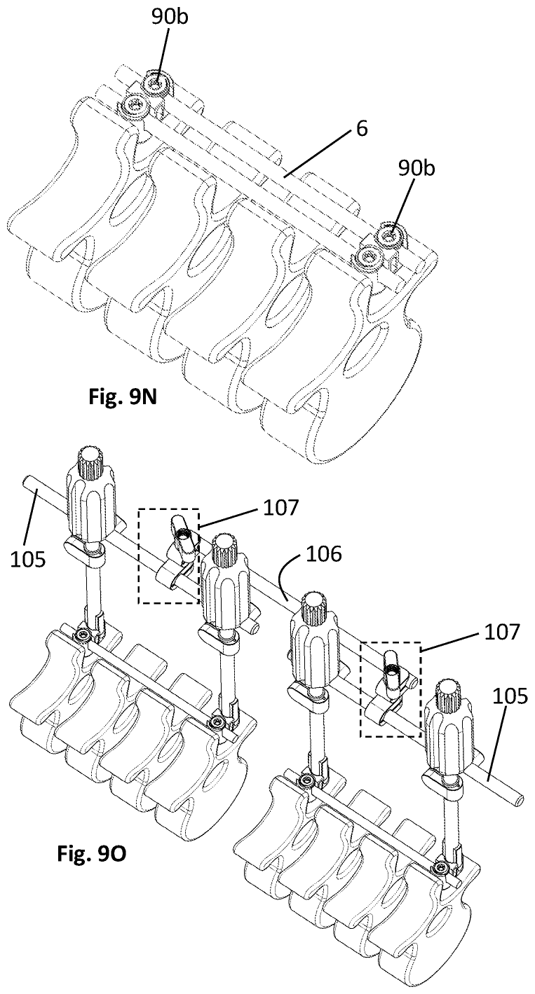

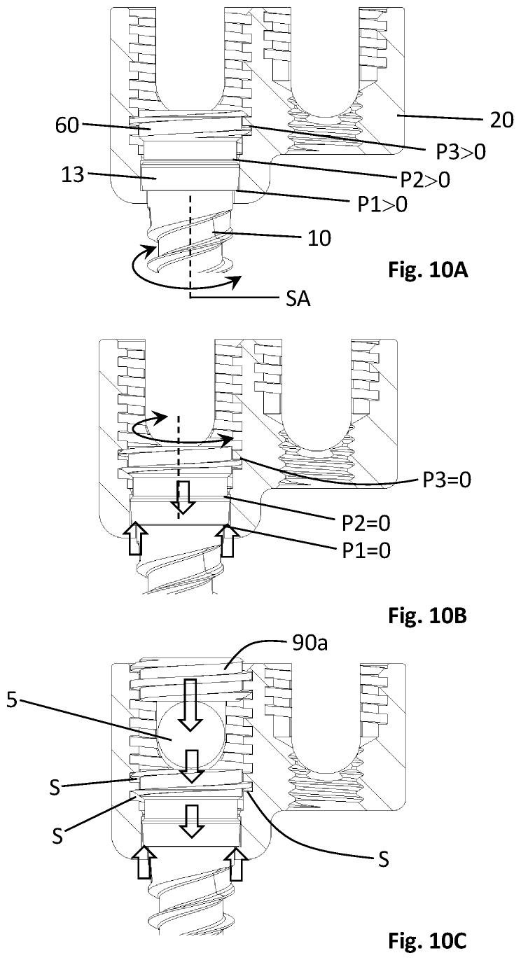

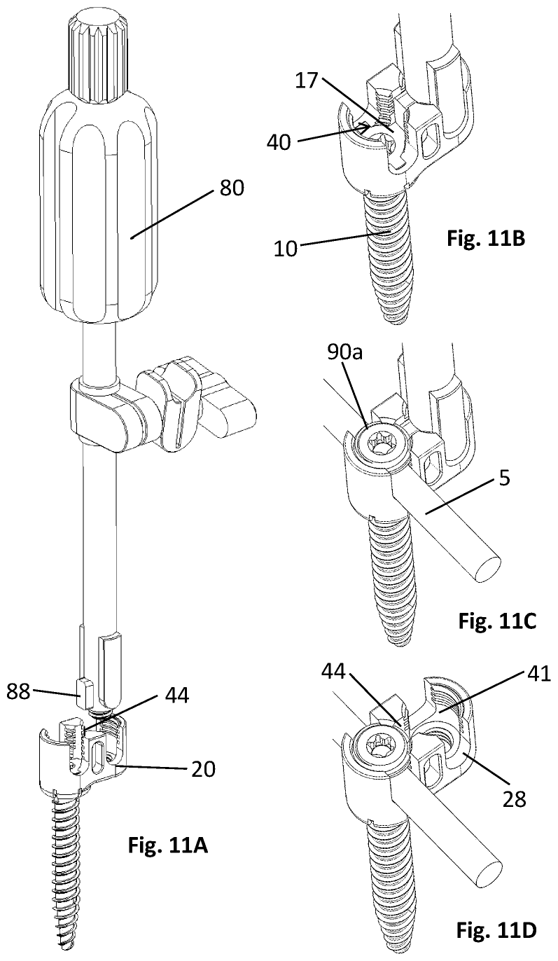

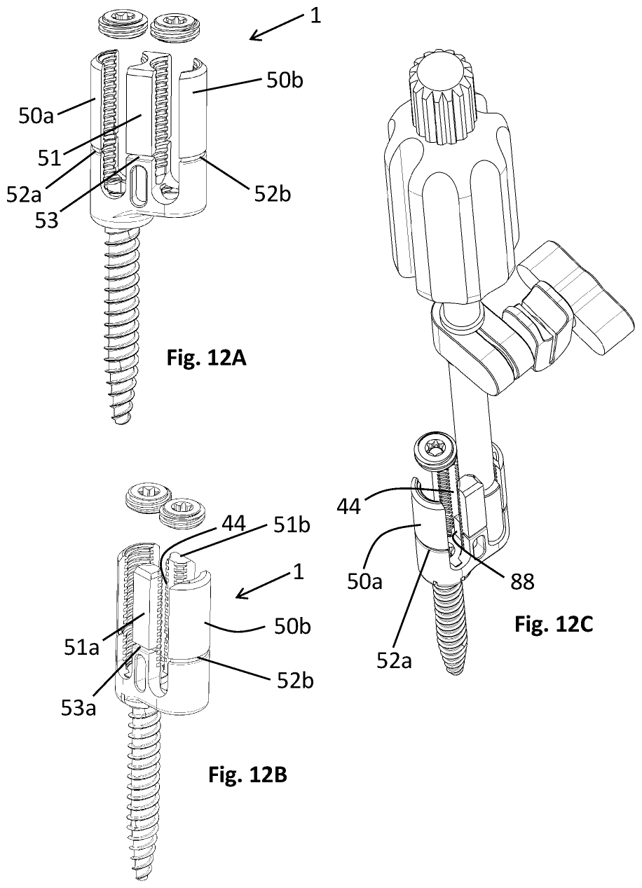

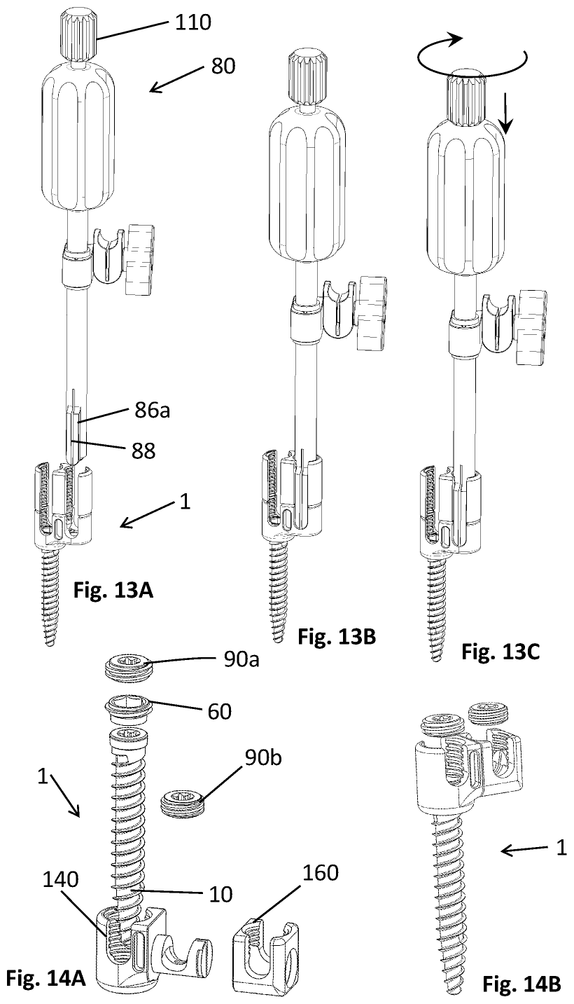

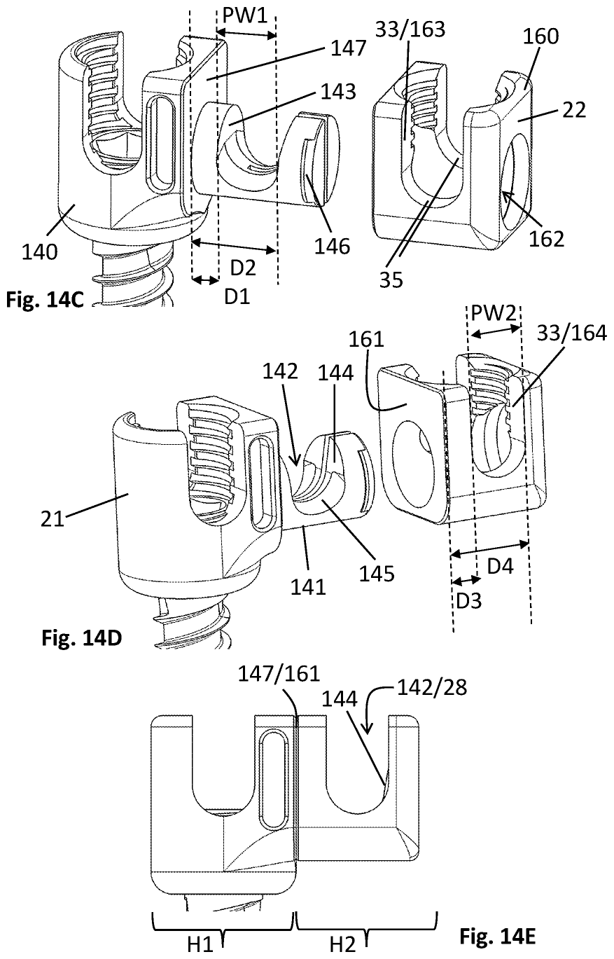

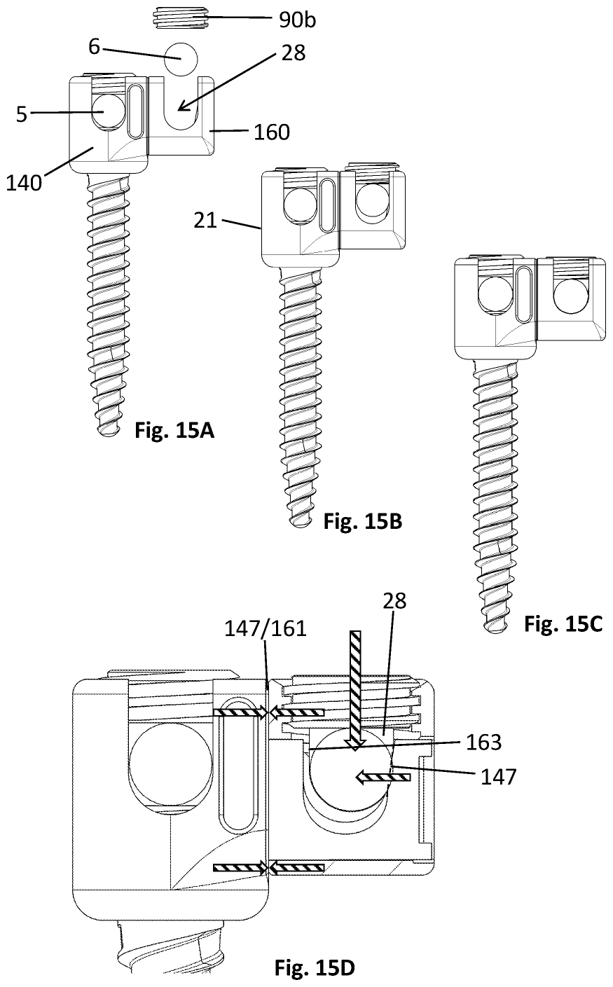

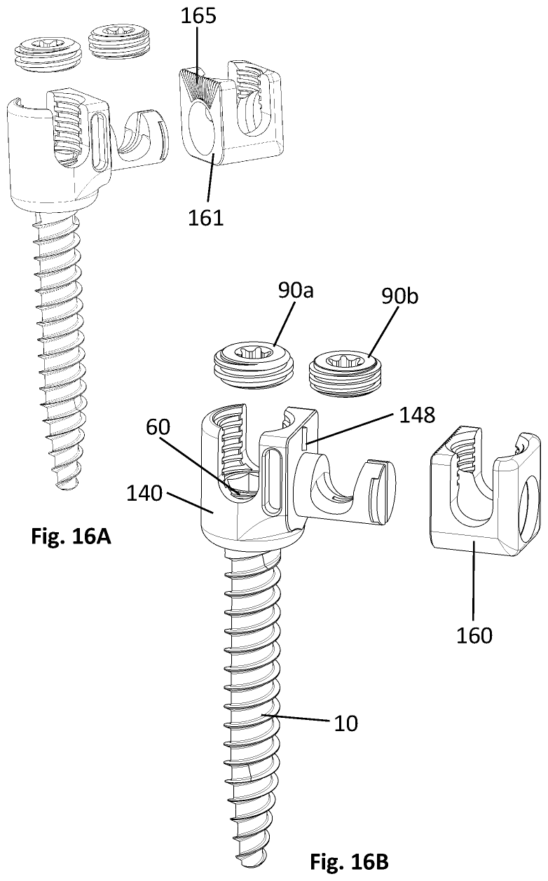

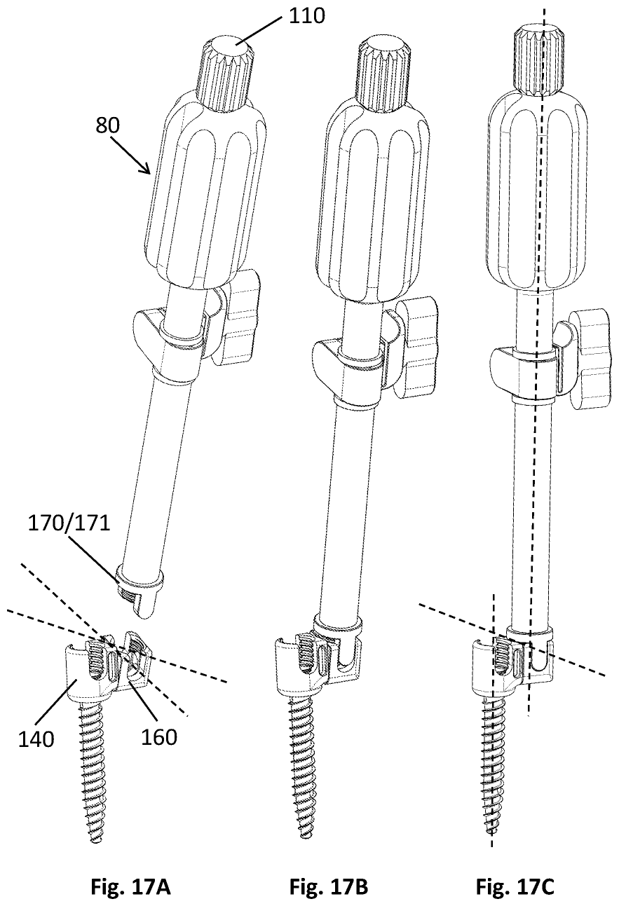

Referring to FIGS. 3A to 3D, the rod receiving head 20 is shown in greater detail. The rod receiving head has a (horizontal) head length (HL), a (horizontal) head width (HW) and a (vertical) head height (HH), which in this example are dimensions orthogonal to each other, where
the head width (HW) and head height (HH) define a first head end 21 and an opposite, second head end 22, which are longitudinally at opposite ends of the connector head,
the head length (HL) and head height (HH) define a first head side 23 and an opposite, second head side 24, and
the head length (HL) and head width (HW) define a head top side 25 and an opposite, head bottom side 26.

A head length (HL) to head width (HW) ratio (HL:HW) may be at least 1:1.5, and more specifically at least 1:1.8. The rod receiving head is depicted as a substantially block-shaped body, having rounded edges. It is to be noted that in order to reduce any soft tissue irritation, implant portions, such as the rod receiving head, have rounded, chamfered or broken edges. The rod receiving head 20 further comprises at least a first rod receiving passage 27 and a second rod receiving passage 28 extending between the first head side 23 and the second head side 24, and which in this example are at least partially open to the head top side 25. In this example, the first and second rod receiving passages are longitudinal openings and they extend parallel or substantially parallel with respect to each other. The rod receiving head 20 is virtually divided into a first half (H1) and a second half (H2) comprising the first and second rod receiving passages, respectively. Thus, the rod receiving passages 27, 28 form an opening to the head top side 25 from where the rods can be inserted into the passages, prior to placement and tightening of the rod fasteners. More specifically, the rod receiving passages comprise a passage start section 29a, 29b, and a passage end section 30a, 30b. In the present example, the passage end sections are sized and shaped to encompass a rod 5, 6 at three sides. In this example the first and second rod receiving passages comprise first side boundaries 32, and second side boundaries, respectively, which are configured to limit the movement of the respective rod. In the present example, the side boundaries form a direct engagement area or surface to limit the movement of the rod. Moreover, the passages comprise a first and second bottom boundary 34, 35.

As depicted in FIGS. 1A to 1C, the bone fastener 10 is assembled into the rod receiving head through the head top side 25 so that the bone fastener shaft 11 protrudes through the head bottom side 26. In order to receive the bone fastener, and more specifically the bone fastener head 13, the rod receiving head comprises a bone fastener head receiving recess 37, which is open to the head bottom side 26. In this example, only the first half (H1) comprises the bone fastener head receiving recess 37, although only the second half (H2) could comprise the bone fastener head receiving recess 37, or both the first and second halves could comprise the bone fastener head receiving recess 37. The head receiving recess 37 may be sized and shaped to directly receive the bone fastener head 13 or a portion of it, and/or it may receive an intermediate inlay (not shown), which is configured to directly receive the bone fastener head.

As depicted in FIGS. 3A to 3C, the head receiving recess 37 is configured as a stepped recess, more specifically as a stepped bore, including a recess top portion 38 and a recess bottom portion 39. The recess top portion is configured to encompass the bone fastener head periphery, where the recess bottom portion 39 is configured to engage directly with the cylindrical head bottom face 18, and forms a seat and inhibits separation of the bone fastener head out of the stepped bore.

The rod receiving head further comprises a first pocket 40, and a second pocket 41, which intersect the first rod receiving passage 27, and the second rod receiving passage 28, respectively. As shown, the first pocket extends from the head top side 25 towards the head bottom side 26 and intersect the head receiving recess 37. The stepped recess extends into the head bottom side, thus forming an opening for the elongated shaft 11. The recess bottom portion 39 has a periphery diameter P∅. In one example, the periphery diameter P∅ is smaller than the head diameter H∅ and greater than the neck diameter N∅ of the bone fastener 10, and so inhibits separation of the bone fastener in at least one direction. It is to be noted that the purpose of the head receiving recess is to encompass and guide the bone fastener in a substantially play-free manner. Alternatively to a cylindrical bore, other shapes may encompass the bone fastener head or shaft. In theory, many recess shapes, preferably providing at least three engagement areas, can for example guide the cylindrical head or shaft. The cylindrical head or shaft can be guided by a triangular recess, quadratic recess, or a multi-facetted recess. Reversely, also a quadratic head can be guided for example by at least partially spherical recess.

At the head top side or adjacent to it, the rod receiving head comprises at least one first locking feature or means 36a, and at least one second locking feature or means 36b, sized and shaped to receive and/or engage with at least one rod fastener 90a, 90b. In the present example, the locking means are configured as a first internal thread or thread portion 42 and a second internal thread or thread portion 43, which intersect the first and second pockets 40, 41, respectively.

In the present example, the first pocket 40 is sized and shaped to receive the locking insert 60. In the design shown in FIGS. 3A to 3D, the first locking means 36a extends from the head top side 25 or head top side region towards and ends near the recess bottom portion 39. Alternatively, the engagement area for the locking insert may be formed by a third locking means 36c, for example a smaller different thread. The internal thread portion 42 of the first pocket 40 is sized and shaped to receive both the locking insert 60 and one rod fastener 90. The internal thread, which is essentially configured as a helical groove, has a first pitch P1, a first crest width CW1, a first thread depth TD1, and a first groove width GW1, as shown in FIG. 3C.

As described, the second pocket 41 intersects the second rod receiving passage 28 and/or the second locking means 36b, which extends from the head top side 25 or head top side region towards the head bottom side 26. The second rod receiving passage is sized and shaped to receive a second rod 6 and upon insertion of a second rod fastener 90b, any movement of the rod in relation to the second rod receiving passage is inhibited. Moreover, the second pocket 41 including integrated features, provides an attachment area or surface for a correction instrument 80 (shown in FIG. 6A). A first purpose of the correction instrument is to form a rigid extension of the screw, and so allow the surgeon to apply greater forces to correct the spinal deformity. Thus, in the present example, the second pocket 41 is sized and shaped to receive at least a portion of a first coupling 84 (shown in FIG. 6A) of the correction instrument 80, and it includes a first connection element or means 45 inhibiting separation of the two. As depicted, the first connection means 45 may be sized and shaped as a threaded connection feature or means 45 at the bottom of the second pocket. Alternatively, the second locking means can be used as a connection area or surface.

FIGS. 4A and 4B illustrate the locking insert 60 in greater detail. The locking insert 60 comprises a locking insert body 61 having a locking insert top side 62 and a locking insert bottom side 63. A locking insert drive 64 extends into the body and is intended to engage with a first locking tool 130, such as a screwdriver or wrench, providing a means to lock the locking insert into the elongated connector head. Furthermore, a locking insert passage 67 passes through the locking insert body from the top side to the bottom side. As depicted, the locking insert passage may at least partially be combined with the locking insert drive 64. The drive and the passage may also be separate elements. The locking insert passage 67 forms an opening through which a screwdriver can be inserted into the first drive 16 of the bone fastener. In this example, the locking insert comprises a first external locking feature or means 65, which is shaped as a first external thread 66. Furthermore, in this example, the locking insert top side 62 is flat, configured to engage against a posterior rod 5, 6 and the locking insert bottom side 63 is configured to engage against the top face 17 of the bone fastener 10. The first external thread, which in this example is essentially configured as a helical groove, comprises a second pitch P2, a second crest width CW2 and a second groove width GW2. In one specific example, the second crest width is significantly smaller than the first groove width GW1 of the first internal thread portion 42.

FIGS. 5A and 5B illustrate one of the rod fasteners 90a in greater detail. The rod fastener 90a comprises a rod fastener body 91 having a rod fastener top side 92 and a rod fastener bottom side 93. A rod fastener drive 94 extends into the body and is intended to engage with a second locking tool such as a screwdriver, providing a means to lock the rod fastener into the elongated connector head. In this example, the rod fastener comprises a second external locking feature or means 95, which is shaped as a second external thread 96. Furthermore, in this example, the rod fastener bottom side 93 is flat, configured to engage against a posterior rod 5, 6.

FIGS. 6A to 6D, 7, and 8 describe multiple instruments that may be part of the kit and/or method. FIGS. 6A to 6D depict an example correction instrument assembly 80. The correction instrument assembly comprises a correction handle 81 and a fixation tool 110. The main correction handle comprises a first grip 82 at a first end 83 and a first coupling 84 at a second end 85. The first coupling is sized and shaped to engage in or with the second pocket 41. In the present example, at least one protrusion 86a, 86b extends away from the correction handle, and it is configured to engage in or with the second rod receiving passage 28 and intended to block a rotational degree of freedom between the rod receiving head and the correction handle. The correction handle further comprises a first channel 87 extending longitudinally along the handle, which is sized and shaped to receive the fixation tool. The fixation tool 110 comprises a fixation tool shaft 111 having a second grip 113 at a first end, and a second, coupling end 112, which is configured to engage with the first connection means 45. In the present example the engagement is made by means of complementary threads. Moreover, the correction handle 81 comprises a universal clamping or joint element 100. As described later, the clamping element or section is used to link more correction handles by means of a stabilisation bar 105 (shown in FIG. 9I). The clamping element is built of multiple hinges 101 and clamps 102 for holding the stabilisation bar. In the present example, the clamping element 100 comprises a wing nut 103, which allows all hinges and clamps to be locked in relation to each other.

FIG. 7 depicts an insertion tool 120 for inserting the spinal bone fastener assembly into a target bone. In the present example, the insertion tool 120 is a screwdriver and has an insertion tool shaft 121, an insertion tool grip 122 at one end, and an insertion tool drive 123 at the other end.

FIG. 8 depicts a locking tool 130 for locking the locking insert 60. In the present example, the locking tool 130 has a locking tool shaft 131, a locking tool grip 131 for manipulation at one end, and a locking tool drive 133 at the other end. The locking tool shaft further comprises a second channel 134 extending longitudinally along and through the handle, which is sized and shaped to receive the insertion tool shaft.

FIGS. 9A to 9O show example surgical steps illustrating the handling and locking principle of the bone fastener assembly 1 and its instruments. For illustration purposes, four bony segments or vertebrae of the spine are shown without any soft tissue such as muscles, intervertebral discs, nerves, etc. FIGS. 9A and 9B depict a pathological situation, where four spinal vertebrae are severely misaligned due to a deformity or a trauma. In the present example, in order to correct the spinal column, two bone fastener assemblies 1 are inserted in the two outer vertebrae. By inserting the insertion tool 120 into the drive 16 of the bone fastener 10, an insertion torque is applied to the bone fastener and the bone fastener assemblies 1 can in this manner be fixated with the vertebrae.

FIG. 9C depicts the insertion of an alignment rod 8 into the second rod receiving passages 28 of the rod receiving heads. The rod receiving heads can rotate freely around the shaft axis SA of the bone fastener. By placing the alignment rod 8, the rod receiving heads 20 are oriented in a desired orientation. In the present example, where the alignment rod is straight, the rod receiving heads 20 are oriented in a parallel manner. FIG. 9C further shows the placement of the locking tool 130. The locking tool is engaged with the locking insert drive 64. FIG. 9D depicts the locking tool 130 in place and the insertion of the insertion tool 120 through the second channel 134. The screwdriver drive 123 of the insertion tool is engaged with the first drive 16 of the bone fastener. By means of holding the insertion tool 120 in a static position, and turning the locking tool 130, the locking insert 60 is tightened against the top face 17 of the bone fastener 10. The rod receiving head, the bone fastener and the locking insert are thus blocked with respect to each other. It is to be noted that the locking insert can also be tightened without countering the applied torque by means of statically holding the insertion tool. By countering the torque, no torque is transferred to the bone fastener, and the bone fastener remains in its initial position. FIGS. 9E and 9F show the above steps being repeated for the second bone fastener assembly.

FIG. 9G depicts a next surgical step, where the correction instruments 80 are assembled into the second pocket 41 of the rod receiving head. The first and second protrusions 86a, 86b are engaged with the second rod receiving passage 28 and simultaneously the second coupling end 112 of the fixation tool is engaged with the first connection means 45. The correction instrument is rigidly fixated to the bone fastener assembly. Manipulating forces that are exerted to the correction instruments are directly transferred to the bone fastener and to the target bones.

FIGS. 9H and 9I depict the placement of a first stabilisation bar 105. The first stabilisation bar 105 is clicked into the clamps 102 of the universal clamping elements 100. In the present example, at this stage, the hinges 101 of the universal clamping element can rotate freely. Furthermore the first stabilisation bar can translate within the clamps. FIG. 9J depicts the correction or realignment step. By handling the correction instruments, the vertebral bodies or spinal column is realigned. The correction instruments help the surgeon to exert high forces for this alignment step. In a next step, the wing nuts 103 are tightened and the stabilisation bar 105 is fixated and the whole construct becomes rigid. The instruments hold the planned correction in place.

Referring to FIGS. 9K and 9L, the placement of a rod 5 into the rod receiving head is shown. The implant is inserted into the first rod receiving passage 27. In a next step the rod fasteners 90a are placed on top of the rod and engaged with the first locking means 36a. As described in greater detail later with reference to FIGS. 10A to 10C, in the present example, the rod fastener 90a is seated against the rod 5, and the rod 5 is seated against the locking insert.

Referring to FIG. 9M, the construct including one rod after removal of the correction instruments is shown. FIG. 9N shows the placement of a second rod. The second rod is inserted into the second rod receiving passage and locked with the second rod fasteners 90b. FIG. 9O depicts how the method as described in connection with FIGS. 9A to 9N may also be applied for a longer posterior construct to correct the spine. In the example of FIG. 9O, two independent corrected areas are connected by a second stabilisation bar 106. The first stabilisation bar 105 is linked to the second stabilisation bar by connecting clamps 107.

Referring to FIGS. 10A to 10C, the interaction of the rod fastener 90a, the rod 5, the locking insert 60, the rod receiving head 20 and the bone fastener 10 is shown in greater detail in cross sectional views. For illustration purposes, the target bones and the instruments are not shown. FIG. 10A shows a first state, where the bone fastener is free to rotate. The locking insert 60 is in an untightened configuration. The bone fastener can rotate around its shaft axis SA. More specifically, a first play P1 exists between the bone faster bottom face 18 and the recess bottom portion 39. Furthermore, a second play P2 exists between the locking insert bottom side 63 and the bone fastener top face 17. A third play P3 exists between the first locking means 36a and the first external thread 66.

FIG. 10B shows a second state, where all play has been eliminated by tightening the locking insert 60. The tightening forces of the locking insert are transferred to the bone fastener head 13 which respectively transfers the tightening forces to the recess bottom portion of the rod receiving head. In the present example, as described in connection with FIGS. 3A to 3C, 4A and 4B, the first external thread 66 has a second crest width CW2, which is significantly smaller than the first groove width GW1 of the first internal thread portion 42. Thus, in the second state, a space S is present between the internal and external threads. Moreover, in the second state, the locking insert top side extends above or over the first bottom boundary 34. FIG. 10C shows a third state, where the rod 5 is inserted and locked by a rod fastener. The rod fastener presses against the rod, the rod presses against the locking insert, the locking insert presses against the bone fastener and the bone fastener seats against the recess bottom portion of the rod receiving head. The space S provides a vertical translation flexibility to the locking insert for a complete force transfer from the rod fastener towards the bone fastener. It is to be noted that the internal and external threads could be reversed on the locking insert and in the first pocket for instance. In other words, the locking insert could have an internal thread and the pocket could comprise an external thread.

FIGS. 11A to 11D show an alternative method of locking the bone fastener in relation to the rod receiving head. The correction instrument 80 comprises a nose 88 which protrudes from the second end 85. The nose is sized and shaped to engage against the bone fastener head 13, and more specifically against its top face 17. It is to be noted that the top face 17 of the bone fastener extends over the first bottom boundary 34. FIGS. 11A and 11B depict the step of inserting the correction instrument into the second pocket 41 of the rod receiving head. The first and second protrusions 86a, 86b are engaged with the second rod receiving passage 28 and simultaneously the second coupling end 112 of the fixation tool is engaged with the first connection means 45. A connecting passage 44 connects the first and second pockets 40, 41 and forms a connecting passage for the nose. The nose 88 presses against the bone fastener head and inhibits its rotation. FIG. 11C depicts the placement of the rod 5 and its fixation by means of a rod fastener. The tightening forces of the rod fastener are transferred to the rod, which transfers the tightening forces to the bone fastener head, which further transfers the tightening forces to the recess bottom portion of the rod receiving head. At this moment rotation of the bone fastener head is inhibited by simultaneous compression of the correction instrument nose and the rod. FIG. 11D depicts the bone screw assembly after removal of the correction instrument. The second rod receiving passage is open to receive a second rod 6 and a second rod fastener 90b.

FIGS. 12A to 12C depict an alternative design of the bone fastener assembly 1, where the rod receiving head 20 comprises at least one vertically directed or oriented side head extension and/or at least one vertically directed or oriented central head extension. The extended head may be provided for implantation purposes and simplified rod alignment handling. After a spinal posterior system has been implanted, these extensions need to be removed. To allow the surgeon to remove the extensions, they comprise a pre-manufactured breaking reliefs or regions most commonly defined by a groove. In the example shown in FIG. 12A, the extended rod receiving head essentially corresponds to the design of FIGS. 3A to 3C. The head comprises two side head extensions 50a, 50b and one central head extension 51. By means of the side breaking reliefs 52a, 52b and the central breaking relief 53, the head can be shortened after insertion and fixation of the rods. In the example shown in FIGS. 12B and 12C, the extended rod receiving head essentially corresponds to the design of FIGS. 11A to 11C. The head comprises two side head extensions 50a, 50b and two central head extensions 51a, 51b. The central head extension is divided in two parts to provide a central passage 44 for the nose 88 of the correction instrument 80. By means of the side breaking reliefs 52a, 52b and the two central breaking reliefs 53a, the head can be shortened after insertion and fixation of the rods.

FIGS. 13A to 13C show an alternative design for the correction instrument, and more specifically for the first coupling 84. In this example, the first coupling 84 comprises a slot 88 extending through at least one of the protrusions 86a and 86b, in this example parallel to the longitudinal axis of the correction instrument. The slot makes the first coupling 84 elastic or compliant. Due to the elasticity, upon insertion of the correction instrument into the rod receiving passage, an initial stability will be present, prior to the final tightening of the fixation tool 110.

FIGS. 14A to 14E show a variant of the bone fastener assembly 1. In this example, the rod receiving head is divided into two portions, which can rotate in relation to each other, and thus the orientation of the second rod receiving passage can be adjusted. FIG. 14A depicts the individual elements of the spinal bone fastener assembly 1 in an exploded view. The bone fastener assembly comprises a bone fastener 10, or more specifically a pedicle screw, a first rod receiving head portion 140, a rotatably coupled second rod receiving head portion 160, a bone fastener engaging locking insert 60, and at least one rod fastener 90a, 90b, such as a set screw. The bone fastener assembly is configured to receive at least one, more preferably two state-of-the-art spinal posterior stabilisation rods 5, 6.

FIG. 14B shows the bone fastener assembly in an assembled configuration. In the assembled configuration, the first and second rod receiving head portions comprise multiple structural elements, which are equivalent to the corresponding elements of the rod receiving head 20 of FIGS. 3A to 3C. Equivalent to the rod receiving head 20, the assembled first and second rod receiving head portions 140, 160 in combination comprise a first head end 21, and a second head end 22 defining the head length HL. With reference to the head length HL, the substantially centrally located area, where both head portions engage or meet, is formed by a first side wall 147 and a second, opposite side wall 161, as described in greater detail later. In the present example, substantially centrally is understood to mean the mid third of the head length HL. The engagement area substantially overlaps with the virtual division into a first half and a second half H1, H2 as described in connection with FIGS. 3A to 3C. Furthermore, in the present example, the bone fastener 10, the first rod receiving head portion 140 including the first rod receiving passage 27, the locking insert 60, the rod 5 and the rod fastener 90a lockingly interact as described with reference to FIGS. 11A to 11C.

FIGS. 14C and 14D show the first and second rod receiving head portions in greater detail. The first rod receiving head portion 140 comprises a projection 141 sized and shaped to receive the second rod receiving head portion 160 in a rotatable manner. In the present example, the projection 141 has a substantially cylindrical shape or outer contour, and it extends orthogonally or substantially orthogonally to the shaft axis SA. A third rod receiving passage 142 extends through the projection, in this example parallel or substantially parallel to the first rod receiving passage 27. The third rod receiving passage comprises a first inner side boundary 143, a first outer side boundary 144 and a third bottom boundary 145. As described, the head portions engage at the first side wall 147. The first inner and outer side boundaries are spaced at a first distance D1, and a second distance D2 from the first side wall 147, respectively. Furthermore, the first inner and outer side boundaries are spaced apart by a first passage width PW1. The end of the projection 141 is sized and shaped as a snap-fit attachment connection 146 to hold the second rod receiving head portion.

The second rod receiving head portion is formed as a block-shaped portion to hold the second rod. The second rod receiving head portion extends between the second side wall 161 and the second head end 22 and has a similar size as the second half H2 shown in FIGS. 3A to 3C. The second head portion comprises a through bore or opening 162 sized and shaped to engage over or with the projection 141 and to snap over the snap-fit attachment 146. As described, the second head portion comprises a second rod receiving passage 28 having second side boundaries 33 and second bottom boundaries 35. In this example, the side boundaries are referred to as second inner side boundaries 163 and second outer side boundaries 164. The second inner and outer side boundaries are spaced at a third distance D3, and a fourth distance D4 from the second side wall 161, respectively. Moreover, the second inner and outer side boundaries are spaced apart by a second passage width PW2. In the present example, the second passage width PW2 is minimally oversized in relation to the rod diameter it is intended to be used with. FIG. 14E shows a detailed view of the assembled head portions in a side view. It is to be noted that in the shown configuration, the head portions 140, 160 are rotatably freely engaged with each other. The first and second side walls 147, 161 are practically engaged, but there is only little friction between them. As depicted, the first outer side boundary 144 overlaps the second rod receiving passage 28. In other words, the first outer side boundary 144 protrudes or extends into the second rod receiving passage 28.

FIGS. 15A to 15D show the principle of locking the first head portion 140 in relation the second head portion 160. For illustration purposes, the locking is shown in a frontal view with parallel rods 5, 6. The same locking principle applies when the rods are oriented in a non-parallel manner. FIG. 15A depicts the insertion of the second rod 6 into the second rod passage 28 and the placement of the second rod fastener 90b. FIG. 15B depicts the moment of engagement of the second rod 6 with the first outer side boundary 144 of the third rod receiving passage 142. At this moment, the rod has not reached the end position, or in other words, it is not seated against the second bottom boundary 35. In this position, the second rod 6 is also engaged with the second inner side boundary of the second rod receiving passage. Now as shown in FIG. 15C, upon further advancing the rod into the rod receiving passage, the rod is pressed sideways (towards the first head end 21) while being engaged with the second inner side boundary 163 of the second rod receiving passage 28. Hence the second rod receiving head portion 160 is pressed sideways. As a result, the first and second side walls 147, 161 are forced to mate in a forceful manner. Rotation of the rod receiving head portions in relation to each other is thus inhibited.

As previously described, the first inner and outer side boundaries 143, 144 are spaced at a first distance D1, and a second distance D2 from the first side wall 147, respectively. Furthermore, the first inner and outer side boundaries are spaced apart by a first passage width PW1. Moreover, the second inner and outer side boundaries 163, 163 are spaced at a third distance D3, and a fourth distance D4 from the second side wall 161, respectively, and the second inner and outer side boundaries are spaced apart by a second passage width PW2. For the locking principle as described in this example, following dimensional relations are valid: PW1 is equal to, or greater than PW2; D1 is smaller than D3; and D2 is greater than D4.

FIGS. 16A and 16B show an alternative variant of the previously described locking mechanism. In this example, the first and second side walls 147, 161 comprise first meshing teeth 148 and second meshing teeth 165, respectively, providing a form-fit locking upon engagement of the sidewalls.

Referring to FIGS. 17A to 17C, the bone fastener assembly of FIGS. 15A to 15D is shown in combination with the correction instrument 80. In this example, the correction instrument comprises an alignment means 170, or more specifically an alignment foot 171. FIG. 17A depicts the bone fastener assembly of FIGS. 14A to 14E, 15A to 15D, and 16A to 16b, where the first rod receiving head portion and the second rod receiving head portion are in an unaligned state. FIGS. 17B and 17C depict the step of placing the correction instrument into the second pocket 41 of the second rod receiving head portion 160. Upon tightening the fixation tool 110, the alignment means engages with the head top side 25 of the first rod receiving head portion. In this example, the second coupling end 112 is configured to engage with the second locking means 36b. The engagement forces both head portions to align and the construct to become one rigid structure. Correction forces can effectively be applied to the bone fastener assembly. In the present example, the correction instrument and the bone fastener are oriented substantially parallel with respect to each other.

While the invention has been illustrated and described in detail in the drawings and foregoing description, such illustration and description are to be considered illustrative or exemplary and not restrictive, the invention being not limited to the disclosed embodiments. Other embodiments and variants are understood, and can be achieved by those skilled in the art when carrying out the claimed invention, based on a study of the drawings, the disclosure and the appended claims. New embodiments or variants may be obtained by combining any of the above teachings.

In the claims, the word “comprising” or “including” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A spinal bone fastener assembly for connecting and/or aligning at least a first rod and a second rod, the bone fastener assembly comprising a bone fastener comprising a bone fastener head and a bone fastener shaft, a correction instrument and/or a locking insert, and a rod receiving head having a head length, a head width and a head height, the head width and the head height defining a first head end, and a second, opposite head end, the head length and head height defining a first head side, and a second, opposite head side, the head length and the head width defining a head top side and, a head bottom side, the rod receiving head comprising:
a first pocket comprising a first locking feature extending towards the head bottom side, and for engaging with a first rod fastener, the first pocket comprising a bone fastener head receiving recess configured to receive the bone fastener head in a first, rotatably unlocked state, and in a second, rotatably locked state;
a second pocket comprising a second locking feature extending towards the head bottom side, and for engaging with a second rod fastener;
a first rod receiving passage extending between the first and second head sides, intersecting the first pocket, and configured to receive the first rod;
a second rod receiving passage extending between the first and second head sides, intersecting the second pocket, and configured to receive the second rod,
wherein the locking insert is configured to engage with the first pocket and/or the correction instrument is configured to engage with the second rod receiving passage such that upon engagement, the bone fastener reaches the second, rotatably locked state from the first, rotatably unlocked state, and wherein at least the first rod receiving passage is open to the top side.

2. The spinal bone fastener assembly according to claim 1, wherein the locking insert is configured to be threadedly engaged with the first locking feature or with a third locking feature in the first pocket.

3. The spinal bone fastener assembly according to claim 1 wherein the rod receiving head comprises a connecting passage connecting the first and second pockets, and sized and shaped to provide a correction instrument clearance to allow the correction instrument to engage with the bone fastener head to thereby allow the bone fastener to reach the second, rotatably locked state from the first, rotatably unlocked state.

4. The spinal bone fastener assembly according to claim 1, wherein the rod receiving head comprises at least one side head extension in extension of at least a portion the respective pocket and at least one side breaking relief.

5. The spinal bone fastener assembly according to claim 1, wherein the rod receiving head comprises at least one central head extension in extension of at least a portion the respective pocket, and at least one central breaking relief.

6. The spinal bone fastener assembly according to claim 1, wherein the bone fastener defines a shaft axis, and wherein the bone fastener is mono-axial, and configured to rotate only around the shaft axis.

7. The spinal bone fastener assembly according to claim 1, wherein the rod receiving head comprises a first rod receiving head portion comprising the first pocket, and a second rod receiving head portion comprising the second pocket, and wherein the first and second rod receiving head portions are configured to rotate with respect to each other.

8. The spinal bone fastener assembly according to claim 7, wherein the first and second rod receiving head portions are configured to rotate with respect to each other around a rotation axis, which is orthogonal or substantially orthogonal to a shaft axis of the bone fastener.

9. The spinal bone fastener assembly according to claim 7, wherein the first rod receiving head portion comprises an attachment mechanism, and a first side wall, and the second rod receiving head portion comprises a second side wall, and wherein the attachment mechanism is configured such that upon placement of the second rod and the second rod fastener in the second pocket while the second rod receiving head portion being engaged with the attachment mechanism, the first and second side walls are arranged to forcefully mate to thereby restrict or inhibit the rotation of the first and second rod receiving head portions with respect to each other.

10. The spinal bone fastener assembly according to claim 9, wherein the attachment mechanism comprises a snap feature for engaging with the second head portion.

11. The spinal bone fastener assembly according to claim 9, wherein the first and second side walls comprise a meshing arrangement.

12. The spinal bone fastener assembly according to claim 7, wherein the first rod receiving head portion comprises an attachment mechanism for engaging with the second rod receiving head portion, and wherein the attachment mechanism comprises a third rod receiving passage comprising a first outer boundary configured to overlap with the second rod receiving passage when the second rod receiving head portion is engaged with the first rod receiving head portion.

13. The spinal bone fastener assembly according to claim 1, wherein the second pocket is sized and shaped to receive at least a portion of the correction instrument.

14. The spinal bone fastener assembly according to claim 1, wherein the rod receiving head is an elongated element, and wherein an axis of elongation of the rod receiving head is orthogonal or substantially orthogonal to a shaft axis of the bone fastener.

15. The spinal bone fastener assembly according to claim 1, wherein the second pocket comprises a threaded connection feature configured to engage with a fixation tool of a correction instrument assembly comprising the correction instrument.

16. The spinal bone fastener assembly according to claim 15, wherein the threaded connection feature is open to the head bottom side.

17. The spinal bone fastener assembly according to claim 1, wherein the first locking feature is configured as an internal thread with a first groove width, and the locking insert comprises an external thread having a crest width such that the groove width is significantly greater than the crest width to leave a space between the internal and external threads when the locking insert is engaged with the first locking feature such that the space provides a vertical translation flexibility for the locking insert for a complete force transfer from the first rod fastener towards the bone fastener when the first rod fastener is engaged in the first pocket and with the first rod.

18. The spinal bone fastener assembly according to claim 1, wherein the rod receiving head comprises a stepped head receiving recess comprising a recess top portion and a recess bottom portion, wherein the recess top portion is configured to encompass the bone fastener head, and wherein the stepped head receiving recess forms a seat for the bone fastener head to inhibit separation of the bone fastener head out of the stepped head receiving recess.

19. A kit comprising at least one bone fastener assembly according to claim 1, wherein the kit further comprises at least one correction instrument sized and shaped to be fixated to the bone fastener assembly in such a manner that at least one of the first and second rod receiving passages is freely accessible for the placement of the first or second rod and the first or second rod fastener.

20. The kit according to claim 19, wherein the correction instrument comprises a universal clamping element sized and shaped to receive a stabilisation bar.

21. The kit according to claim 19, wherein the kit further comprises at least one stabilisation bar connected to the correction instrument.

22. The kit according to claim 19, wherein the kit further comprises an insertion tool for inserting the spinal bone fastener assembly into a target bone and a locking tool for locking the locking insert.

23. The kit according to claim 22, wherein the locking tool comprises a locking tool channel sized and shaped to receive an insertion tool shaft of the insertion tool.

24. The kit according to claim 19, wherein the correction instrument comprises a coupling for engaging with the second rod receiving passages, and wherein the coupling comprises a slot extending through at least one protrusion of the coupling to allow the coupling to flex upon insertion of the correction instrument into the second rod receiving passage.

25. A method of correcting a spinal column by using at least two spinal bone fastener assemblies according to claim 1, wherein the method comprises:
inserting the at least two bone fastener assemblies in two different vertebral bodies;
locking the respective rod receiving head in relation to the respective bone fastener by means of the respective locking insert or the respective correction instrument;
inserting the respective correction instrument in the respective first or second rod receiving passage;
movably connecting the correction instruments by means of a stabilisation bar;
correcting the spinal column;
rigidly securing the stabilisation bar to maintain the correction;
placing and securing a first rod in the respective first or second rod receiving passages;
detaching the correction instruments from the at least two spinal bone fastener assemblies; and
placing and securing a second rod in the respective second or first rod receiving passages.
